(19) United States
(12) Patent Application Publication
(10) Pub. No.: US 2018/0220990 A1
(43) Pub. Date: Aug. 9, 2018

(54) METHOD OF DETECTING VASCULAR CONDITION BASED ON POINT FLUCTUATION CONDUCTION CHARACTERISTICS OF THE HEART AND DEVICE UTILIZING THE SAME

(71) Applicant: GUANGZHOU PHONPAD INFORMATION TECHNOLOGY COOPERATION LIMITED, Guangzhou (CN)

(72) Inventors: Gang WEI, Guangzhou (CN); Jiaojiao LIU, Guangzhou (CN); Yige WANG, Guangzhou (CN); Yan CAO, Guangzhou (CN); Cui YANG, Guangzhou (CN); Biyun MA, Guangzhou (CN); Jie LI, Guangzhou (CN); Mingjian ZHAO, Guangzhou (CN)

(21) Appl. No.: 15/945,212

(22) Filed: Apr. 4, 2018

Related U.S. Application Data
(63) Continuation of application No. PCT/CN2015/092741, filed on Oct. 23, 2015.

(30) Foreign Application Priority Data
Oct. 13, 2015 (CN) 201510658939.1

(51) Int. Cl. A61B 8/08; A61B 8/14; A61B 8/00

(52) U.S. Cl. CPC A61B 8/0883; A61B 8/145; A61B 8/54; A61B 8/0891; A61B 8/4472; A61B 8/4477; A61B 8/46

(57) ABSTRACT

A method of detecting the vascular condition based on point fluctuation conduction characteristics of the heart and a device utilizing the same are disclosed in this patent. The method comprises: attaching flexible detection terminals firmly against the external skin adjacent to the heart or vascular walls; discretizing the detection area of the heart muscle or vascular walls into a plurality of preset detection points; through active emission of ultrasonic waves and continuous array reception of ultrasonic echoes carrying out multidimensional inversion of point fluctuations/point pulse waves using backend inversion; taking the multiple point fluctuations of the heart muscle as the multiple input signals of the vascular system and the multiple point pulse waves of a certain detection location of the blood vessel as the multiple output signals of the vascular system; identifying the vascular conduction characteristics by using digital signal processing methods; obtaining the conduction characteristics of a certain section of the blood vessel by decomposing the vascular conduction characteristics at different locations based on the cascade feature of the blood vessel; through feature extraction and feature comparison realizing the prediction and diagnosis of the vascular condition and the corresponding symptoms. The device comprises a plurality of flexible detection terminals with ultrasonic transceiving capability, a control module and a computing module, wherein the flexible detection terminal includes an ultrasonic transceiver probe array which is fixed on a flexible circuit board.

### FIELD OF THE INVENTION

The present invention relates generally to pulse wave and vascular condition detection technology. More specifically it relates to a device and method that detect vascular condition based on point fluctuation conduction characteristics of the heart.

### BACKGROUND

Cardiovascular disease is a common disease that seriously threatens human health. Routine examinations include blood pressure test, electrocardiogram (ECG), complete blood count, blood cholesterol and blood sugar tests, etc. Among them, ECG makes use of electrodes placed on the skin to record the electrical activity of the heart over a period of time. These electrodes detect the tiny electrical changes on the skin that arise from the heart muscle's electrophysiologic pattern of depolarizing and repolarizing during each heartbeat so as to determine the health condition of the heart. ECG is helpful in diagnosing arrhythmias, myocardial ischemia, myocardial infarction, and other heart diseases. However, the human body is a good conductor of electricity. The electrical signal recorded on the skin is the superposition of the electrical signals generated by the heart muscle after passing through multiple paths. Therefore, it is inaccurate to diagnose heart diseases based on the electrical signal measured on the skin.

Pulse diagnosis is a unique diagnosis method of traditional Chinese medicine. Chinese medicine practitioners, by palpating the patient's radial arteries and sensing the depth, speed, strength, rhythm and shape of the arterial pulse, can infer the cause and condition of the disease and determine the prognosis of the disease according to the obtained pulses. However, these pulses are based on practitioners' finger feeling and thus completely depend on the individual's subjective judgments. The arterial pulse at the wrist and its changes form pulse waves, which are waves of blood pushed from the heart into the circulation with each heartbeat. Therefore, the comprehensive information of the shape, strength, speed, rhythm and other aspects of pulse waves reflects many physiological and pathological features of the cardiovascular system. Currently, noninvasive pulse wave detection methods at home and abroad are mainly divided into two categories, the detection method based on pressure waves and that based on volume waves. Physiological information related to cardiovascular, such as heart rate, cardiac output, vascular compliance, and so on, can be obtained through the waveform analysis of pulse waves. For the detection method based on pressure waves, it directly detects pressure waves generated by contraction and expansion of vascular walls of some superficial arteries. Since it has stringent requirements for testing position, it has relatively large interference in the measurement. For the detection method based on volume waves, volume waves are formed when pressure waves are filtered through the resistance of distal capillaries and multiple leaks of the capillary network. In this case, some cardiovascular information might be lost.

By analyzing the characteristics of pulse waves, we can track, diagnose, and predict certain cardiovascular diseases. The existing detection methods are limited to the analysis of the periodic features and changes of pulse waves (without considering the influence of the signal source) and involve less nonlinear distortion information. The pulse wave velocity determined by the time delay estimation is related to not only the elasticity of vessel walls, but also the caliber, thickness, blood density, blood viscosity, and other factors of arterial vessels. Although the existing detection methods can provide an approximate estimation of arterial elasticity, they can determine neither the location of the disease nor the specific cause of the disease (for example, changes in blood viscosity, angiosclerosis, or other reasons).

Targeting at these problems, this patent proposes a device and method that detect vascular condition based on point fluctuation conduction characteristics of the heart. The device directly detects the beats of the heart muscle and vascular walls through active emission of ultrasound and array reception. Then the vascular system between the heart muscle and vascular walls is identified using digital signal processing methods. Since the device has small radiation, simple structure, high detection precision and is easy and safe to operate, it can be widely used in health monitoring, remote examination, auxiliary diagnosis, and other fields.

### SUMMARY OF THE INVENTION

Heartbeat is the power source of the human blood circulatory system. During systole, the ventricles contract, pumping blood through the body. During diastole, the ventricles relax and fill with blood again. This leads to cyclical changes of arterial blood pressure and arterial volume, which result in cyclical fluctuations of the arterial wall and then form arterial pulse waves. Arterial pulse waves can be perceived in some superficial arteries of the human body. Ventricular pressure rises and falls. The fluctuation of the ventricular pressure is conducted to the great vein close to the heart along the inverse direction of the blood flow, which makes the venous wall expand and contract with the fluctuation of the venous pressure and then forms venous pulse waves. Therefore, the heartbeat is the source of pulse waves of the blood vessel, which corresponds to a continuous solution domain having infinite degrees of freedom. According to the finite element method, the detection area of the heart muscle or vascular walls can be discretized into a plurality of detection points. The locations of these detection points are selected according to certain rules and each detection point corresponds to a beat point. Each beat point in the heart detection area can be considered as a point source that forms pulse waves of the blood vessel, i.e., a point wave source. The position of each point wave source changes with time due to heartbeat, which forms a point fluctuation. Due to the expansion and contraction of blood vessels, the position of each beat point in the detection area of the vascular wall also changes, which forms point pulse waves. From the point of view of signal processing, multiple point pulse waves in the detection area of the vascular wall can be regarded as the diffusion function of multiple point fluctuations in the heart detection area after passing through the vascular system. If multiple point fluctuations in the heart detection area are considered as the multiple input signals to the vascular system, then multiple point pulse waves in the detection area of the vascular wall are the multiple output signals of the vascular system. The point fluctuations of the heart muscle and the point pulse waves of the vascular wall can be obtained through active detection using flexible detection terminals placed on the skin. The vascular conduction characteristics between the multi-input signal and the multi-output signal can be identified using digital signal processing methods and then the vascular features can be extracted and compared.

In order to achieve the above objective, the present invention provides a method for detecting the vascular condition based on point fluctuation conduction characteristics of the heart. In this invention, the flexible detection terminals are fixed on the external skin close to the heart or the vascular wall and firmly against the skin using couplant. An ultrasonic transceiver probe array is set in the flexible detection terminal. The transmission probe converts the electric signal into an ultrasonic signal and emits it. Then reflection occurs at the selected detection points on the heart muscle or vascular walls and a part of the reflected waves are received by adjacent ultrasonic receiving probes. Using echo analysis and geometric inversion, the positions of the detection points that generate reflection can be determined. With continuous detection, time and spatial multidimensional detection can be carried out for the fluctuation formed by the contraction and expansion of the detection point. Then the multiple point fluctuations in the heart detection area and the multiple point pulse waves in the detection area of the vascular wall can be obtained. Take the multiple point fluctuations of the heart as the multiple input signals and multiple point pulse waves of the vascular wall as the multiple output signals. Then the vascular conduction characteristics between the multi-input signal and the multi-output signal can be identified using digital signal processing methods. By decomposing the vascular conduction characteristics based on the cascade feature of the blood vessel, the conduction characteristics of a certain section of the blood vessel can be obtained. Then through feature extraction and feature comparison, detection and auxiliary diagnosis of the vascular condition can be realized; moreover, disease prediction and curative effect tracking could also come true.

The present invention also provides a device that detects vascular condition based on point fluctuation conduction characteristics of the heart. The device includes a plurality of flexible detection terminals with ultrasonic transceiving capability, a control module and a computing module, wherein the flexible detection terminal is connected with the control module, and the control module is connected with the computing module; the flexible detection terminal is responsible for driving and transmitting the ultrasonic detection signal, receiving and preprocessing the ultrasonic echo signal, and data communications; the control module is responsible for setting and controlling detection parameters of the whole device, generating detection signals, storing detection data, comparing vascular conduction characteristics, data communications, and human-computer interaction; the computing module is responsible for inversion of point fluctuation and pulse waves, identification of vascular conduction characteristics, extraction of vascular features, and data communications.

The abovementioned flexible detection terminal includes an ultrasonic transceiver probe array, a transmission processing unit, a reception processing unit, and a communication unit, all of which are fixed on a flexible circuit board and packaged using sound transparent and waterproof material. Flexible detection terminals are fixed by tape on the external skin close to the heart or the vascular wall and firmly against the skin using couplant. In the flexible detection terminal, the ultrasonic transceiver probe array is composed of multiple ultrasonic probes that are arranged according to a certain rule and form an array, wherein one of the probes is responsible for ultrasonic emission and the other probes are responsible for ultrasonic echo reception; the transmission processing unit performs amplification and D/A conversion on the signal received from the control module according to the set parameters and through the transmission probe the obtained electrical signal is converted into an ultrasonic signal, which is then emitted; the reception processing unit performs amplification, A/D conversion and data buffering on the received signal according to the set parameters; the communication unit is responsible for the data and control information communication between the flexible detection terminal and the control module using the wired/wireless communication method.

The abovementioned control module includes a broadband signal generation unit, a master controller, a storage unit, a feature comparison unit, a human-computer interaction unit, a communication unit, and a data buffering unit. The broadband signal generation unit is responsible for generating and driving the broadband detection signal, which is sent to different flexible detection terminals through the communication unit; the master controller is responsible for scheduling among different units to ensure the normal operation of the entire system; the storage unit is in charge of storing the classification feature database, detection locations, vascular conduction features, myocardial point fluctuations, point pulse waves, the patient's symptoms and age, etc.; the feature comparison unit is responsible for carrying out comparison between the vascular conduction characteristics and the feature database: the human-computer interaction unit is responsible for human-computer interaction and can be used for the parameter setting, the input of the control command and the output of the error information; the communication unit is responsible for the data and control information communication between the flexible detection terminal and the control module as well as between the control module and the computing module using the wired/wireless communication method; the data buffering unit is responsible for caching ultrasound echo signals and control parameters.

The abovementioned computing module includes an inversion unit, a conduction characteristic identification unit, a feature extraction unit and a communication unit. Based on the ultrasonic echo signals received by the reception probes of the flexible detection terminal, the inversion unit performs time and spatial time-domain/frequency-domain inversion of the positions and their variations of multiple detection points of the heart muscle or vascular walls in the detection area using frequency estimation algorithms and geometric principles, and obtains multidimensional time-domain/frequency-domain characteristics of multiple point fluctuations of the heart muscle or multiple point pulse waves of the vascular wall. The conduction characteristic identification unit takes the multiple point fluctuations of the heart muscle as the input and the multiple point pulse waves of the vascular wall as the output, identifies the vascular conduction characteristics between the multi-input signal and the multi-output signal using digital signal processing methods, and then decomposes the vascular conduction characteristics based on the cascade feature of the blood vessel to obtain the conduction characteristics of a certain section of the blood vessel. The feature extraction unit is responsible for extracting the features of the vascular conduction characteristics and then obtaining the conduction features of a certain section of the blood vessel. The communication unit is responsible for the data and control information communication between the computing module and the control module using the wired/wireless communication method.

A method for detecting the vascular condition based on point fluctuation conduction characteristics of the heart includes the following steps:

Step 1: Parameter setting of the device.

The set parameters include: locations of the detection points, parameters of the broadband detection signal, parameters of the inversion unit, parameters of the conduction characteristic identification unit, parameters of the feature extraction unit, parameters of the communication unit, and parameters for displaying detection results:

Step 2: Automatic detection of device status.

Device status detected in this step includes: connection status of the probe array of flexible detection terminals, connection status of the flexible detection terminal and the control module, connection status of the control module and the computing module, connection and online status of the communication unit, power capacity status of flexible detection terminals;

Step 3: Generation of the broadband detection signal.

The broadband signal generation unit of the control module generates the broadband detection signal according to the parameters of the broadband detection signal set in Step 1:

Step 4: Ultrasonic wave transmission.

The communication unit of the flexible detection terminal is responsible for receiving the broadband detection signal generated by the control module. The received signal goes through the amplifier and D/A converter in the transmission processing unit, and is converted by the transmission probe into ultrasonic waves, which are then emitted;

Step 5: Ultrasonic echo reception.

The emitted ultrasonic waves are reflected and form ultrasonic echo when encountering the heart muscle or vascular walls. Part of the ultrasonic echo is received by the adjacent ultrasonic probes and the received echo signal is stored in the data buffering unit after going through the amplifier and A/D converter in the reception processing unit and then sent to the control module through the communication unit;

Step 6: Inversion of point fluctuation/point pulse wave;

Perform time and spatial time-domain/frequency-domain inversion of the positions and their variation of multiple detection points of the heart muscle/vascular walls in the detection area using frequency estimation algorithms and geometric principles, and obtain multidimensional time-domain/frequency-domain characteristics of point fluctuations of the heart muscle or point pulse waves of vascular walls;

Step 7: Identification and decomposition of the conduction characteristics.

Take the multiple point fluctuations of the heart muscle as the input and the multiple point pulse waves of the vascular wall as the output and identify the vascular conduction characteristics between the multi-input signal and the multi-output signal using digital signal processing methods. Then obtain the conduction characteristics of a certain section of the blood vessel by decomposing the vascular conduction characteristics based on the cascade feature of the blood vessel;

Step 8: Feature extraction.

By analyzing the vascular conduction characteristics in the time domain/frequency domain, obtain the time-domain/frequency-domain conduction feature of a certain section of the blood vessel;

Step 9: Result storing and classification.

According to the vascular conduction feature, judge the condition of the blood vessel detected and display it on the human-computer interaction platform. Also store the classification feature database, detection locations, vascular conduction features, myocardial point fluctuations, point pulse waves, the patient's symptoms and age, etc., in the storage unit and the detection results are divided into the following three categories and processed separately.

(1) with disease symptoms. The detection results in this category indicate that a certain section of the blood vessel possesses relevant pathological features and matches the feature information in the classification feature database. The feature information of the vascular conduction characteristics will be retained in the feature database of the corresponding symptom in the storage unit;

(2) no disease symptoms. The detection results in this category indicate that a certain section of the blood vessel does not possess relevant pathological features and the data in the storage unit are directly discarded;

(3) suspicious disease symptoms. The detection results in this category cannot provide a definite diagnosis of the vascular condition. Such feature data are stored in the storage unit and when the detection device is available or the test is completed, the testing staff can choose whether to perform repeated tests and more accurate data processing.

The beneficial effect of the invention is as follows.

(1) In the present invention, the detection area of the heart muscle/vascular walls is discretized into a plurality of detection points. Through continuous active detection of the positions and their variations of the detection points, the point fluctuations of the heart muscle and the point pulse waves of the vascular wall can be obtained. Therefore, detection accuracy is improved through continuous detection and rich information is acquired through multi-point detection.

(2) In this invention, an ultrasonic transceiver probe array is set in the flexible detection terminal. Active emission of ultrasonic waves and continuous array reception of ultrasonic echoes are adopted. Time and spatial multidimensional inversion of point fluctuations/point pulse waves of the detection point is carried out using echo analysis, frequency estimation algorithms and geometric principles. The information carried is rich, which improves detection accuracy.

(3) In this invention, the multiple point fluctuations of the heart muscle are taken as the input and the multiple point pulse waves of the vascular wall are taken as the output. Then the vascular conduction characteristics between the input signal and the output signal can be identified using digital signal processing methods. Therefore, the feature information of the blood vessel is rich and complete, which can improve detection accuracy.

(4) The present invention can provide the conduction characteristics of a certain section of the blood vessel by decomposing the vascular conduction characteristics based on the cascade feature of the blood vessel. Through feature extraction and comparison, the condition of a certain section of the blood vessel is obtained, which is helpful to determination and localization of the disease.

(5) The present invention can record detection locations, vascular features, myocardial point fluctuations, point pulse waves, the patient's symptoms and age, etc each time. It can provide a reference for future diagnosis and evidence of curative effect by establishing a database.

(6) The invention has the advantages of low requirements for testing staff, small radiation, simple structure, high detection precision, being easy and safe to operate, etc. Automatic diagnosis and curative effect tracking can be realized based on the data analysis and feature comparison results of the back end.

### BRIEF DESCRIPTION OF THE DRAWINGS

FIG. 1 is a system block diagram of the invention;

FIG. 2 illustrates a flexible detection terminal of the invention;

FIG. 3 is a schematic of the detection process of the invention;

FIG. 4 is a schematic of one embodiment of the invention;

FIG. 5 is a schematic of decomposition of vascular conduction characteristics of the invention;

FIG. 6 is a workflow diagram of the invention.

### DETAILED DESCRIPTION OF THE PREFERRED EMBODIMENTS

The preferred embodiment of the invention is described below with reference to the figures. However, this should not limit the scope of the present invention.

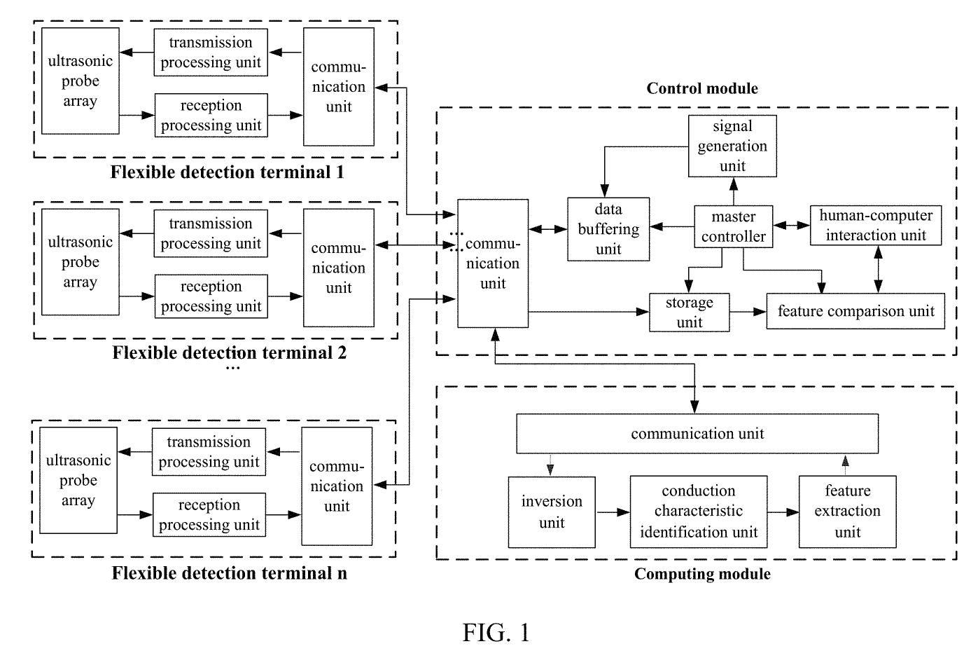

The present invention provides a device that detects vascular condition based on point fluctuation conduction characteristics of the heart, which includes a plurality of flexible detection terminals with ultrasonic transceiving capability, a control module and a computing module. The system block diagram of the invention is shown in FIG. 1. The flexible detection terminal is connected with the control module, and the control module is connected with the computing module; the flexible detection terminal is responsible for driving and transmitting the ultrasonic detection signal, receiving and preprocessing the ultrasonic echo signal, and data communications; the control module is responsible for setting and controlling detection parameters of the whole device, generating detection signals, storing detection data, comparing vascular conduction characteristics, data communications, and human-computer interaction; the computing module is responsible for inversion of point fluctuation and pulse waves, identification of vascular conduction characteristics, extraction of vascular features, and data communications.

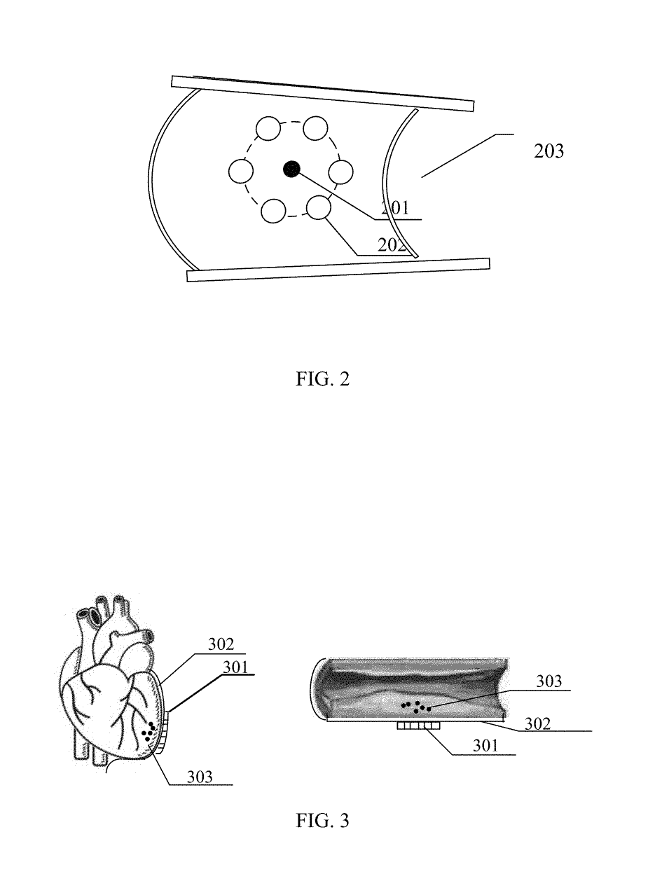

The flexible detection terminal in this embodiment includes an ultrasonic transceiver probe array, a transmission processing unit, a reception processing unit, and a communication unit, all of which are fixed on a flexible circuit board. FIG. 2 illustrates a flexible detection terminal of the invention. The abovementioned ultrasonic transceiver probe array is composed of multiple ultrasonic probes that are arranged according to a certain rule and form an array, wherein the transmission probe 201 in the center is responsible for ultrasonic emission and the reception probes 202 around are responsible for ultrasonic echo reception. The ultrasonic transceiver probe array is fixed on a flexible circuit board 203 and packaged using sound transparent and waterproof material. The detection process of the invention is shown in FIG. 3. During detection, the flexible detection terminal 301 is fixed by tape on 302 the external skin close to the heart or the vascular wall and firmly against the skin using couplant. The positions of 303 the selected detection points are inverted through active emission of ultrasonic waves and array reception of ultrasonic echoes. The abovementioned transmission processing unit performs amplification and D/A conversion on the signal received from the control module according to the set parameters and through the transmission probe the obtained electrical signal is converted into an ultrasonic signal, which is then emitted; the abovementioned reception processing unit performs amplification, A/D conversion and data buffering on the received signal according to the set parameters; the abovementioned communication unit is responsible for the data and control information communication between the flexible detection terminal and the control module using the wired/wireless communication method.

The control module in this embodiment includes a broadband signal generation unit, a master controller, a storage unit, a feature comparison unit, a human-computer interaction unit, a communication unit, and a data buffering unit. The abovementioned broadband signal generation unit is responsible for generating and driving the broadband detection signal, which is sent to different flexible detection terminals through the communication unit; the abovementioned master controller is responsible for scheduling among different units to ensure the normal operation of the entire system; the abovementioned storage unit is in charge of storing the classification feature database, detection locations, vascular conduction features, myocardial point fluctuations, point pulse waves, the patient's symptoms and age, etc.; the abovementioned feature comparison unit is responsible for carrying out comparison between the vascular conduction characteristics and the feature database; the abovementioned human-computer interaction unit is responsible for human-computer interaction and can be used for the parameter setting, the input of the control command and the output of the error information; the abovementioned communication unit is responsible for the data and control information communication between the flexible detection terminal and the control module as well as between the control module and the computing module using the wired/wireless communication method; the abovementioned data buffering unit is responsible for caching ultrasound echo signals and control parameters.

The computing module in this embodiment includes an inversion unit, a conduction characteristic identification unit, a feature extraction unit and a communication unit. Based on the ultrasonic echo signals received by the reception probes of the flexible detection terminal, the abovementioned inversion unit performs time and spatial time-domain/frequency-domain inversion of the positions and their variation of multiple detection points of the heart muscle or vascular walls using frequency estimation algorithms and geometric principles, and obtains multidimensional time-domain/frequency-domain characteristics of multiple point fluctuations of the heart muscle or multiple point pulse waves of the vascular wall; the abovementioned conduction characteristic identification unit takes the multiple point fluctuations of the heart muscle as the input and the multiple point pulse waves of the vascular wall as the output, identifies the vascular conduction characteristics between the multi-input signal and the multi-output signal using digital signal processing methods, and then decomposes the vascular conduction characteristics based on the cascade feature of the blood vessel to obtain the conduction characteristics of a certain section of the blood vessel; the abovementioned feature extraction unit is responsible for extracting the feature of the vascular conduction characteristics and then obtaining the conduction feature of a certain section of the blood vessel; the abovementioned communication unit is responsible for the data and control information communication between the computing module and the control module using the wired/wireless communication method.

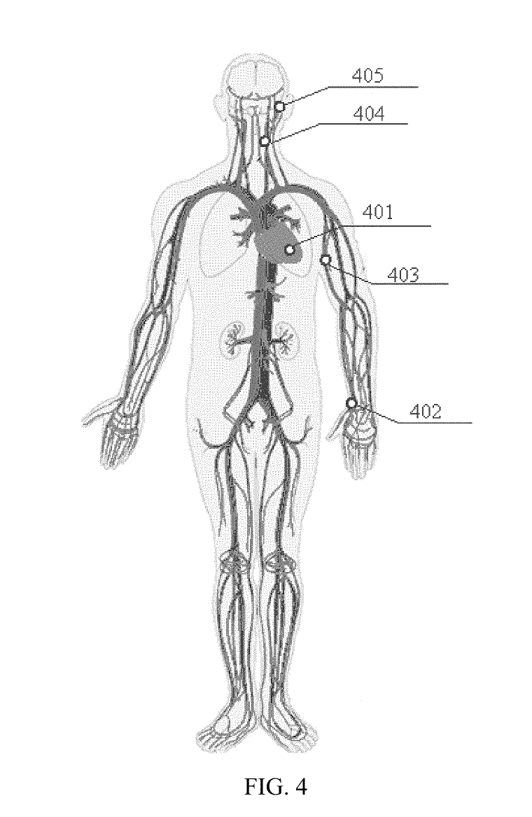

The schematic of this embodiment is shown in FIG. 4. The flexible detection terminals are fixed on the skin that is close to 401 ventricular heart muscle, 402 radial artery, 403 brachial artery, 404 carotid artery, 405 anterior auricular artery, respectively, and cling to the skin using couplant. At these locations, the beats of the heart muscle and the vascular wall are palpable. At each detection location, multiple detection points are selected according to a certain rule. The control module generates broadband detection signals and transmits them to each detection terminal through the communication unit. The signals go through amplification and D/A conversion in the transmission processing unit and through the transmission probe the obtained electrical signals are converted into ultrasonic signals, which are then emitted. Then reflection occurs at the selected detection points on the heart muscle or vascular wall and a part of the reflected waves are received by adjacent ultrasonic receiving probes. The received ultrasonic echoes go through amplification, A/D conversion and data buffering in the reception processing unit. Then the buffered data are sent to the control module through the communication unit. The control module receives the data and after being buffered the data are sent to the computing module. Then the inversion unit performs time and spatial time-domain/frequency-domain inversion of the positions and their variation of multiple detection points of the heart muscle or vascular wall using frequency estimation algorithms and geometric principles, and obtains multiple point fluctuations or multiple point pulse waves. The obtained point fluctuations or multiple point pulse waves are then sent to the conduction characteristic identification unit, which takes the multiple point fluctuations of the heart muscle as the input and the multiple point pulse waves of the vascular wall as the output, and identifies the vascular conduction characteristics between the ventricle and the radial artery, between the ventricle and the brachial artery, between the ventricle and the carotid artery, between the ventricle and the anterior auricular artery, respectively, using digital signal processing methods. Next, based on the cascade feature of the blood vessel, decompose the abovementioned vascular conduction characteristics to obtain the arterial conduction characteristics between the wrist and the upper arm and between the neck and the anterior ear, respectively. In the feature extraction unit, extract the time and frequency feature of the vascular conduction characteristics. The obtained conduction feature is sent to the control module through the communication unit and stored in the storage unit. The master controller compares the received feature information with those in the feature database and diagnoses the vascular condition and disease symptoms, such as angiosteosis, thrombus, angioma, etc. It can also carry out curative effect tracking.

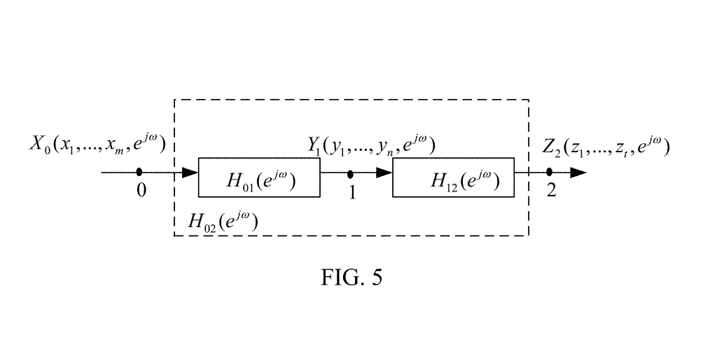

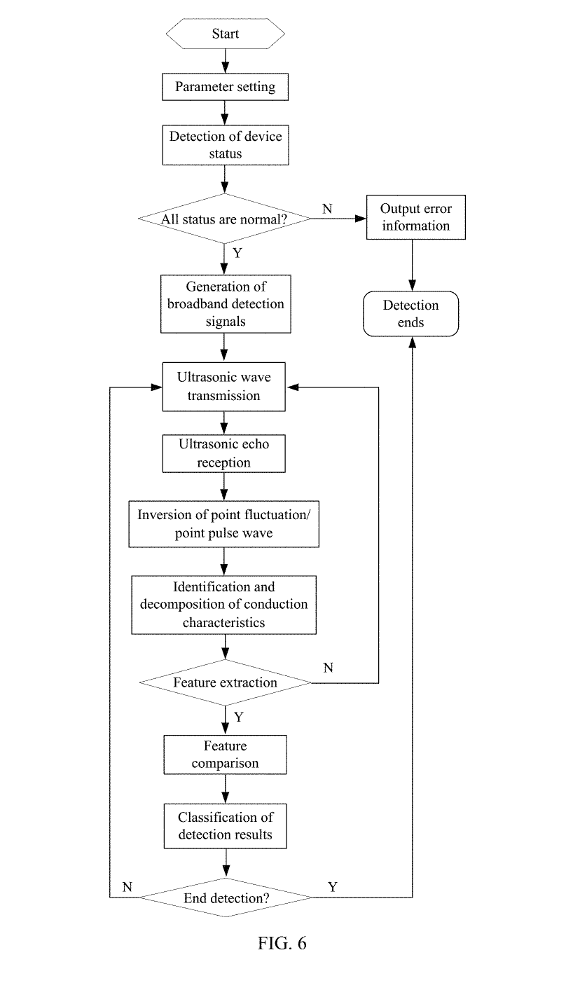

FIG. 5 is a schematic of the decomposition of vascular conduction characteristics of the present invention.

Set a plurality of detection points x₁, . . . , xₘ at detection location 0 of the ventricle. Detected signals from multiple detection points are used as the multi-input signals at this location, i.e., multiple point fluctuation signals, denoted by X(x₁, . . . , xₘ,e^{jω}) Set a plurality of detection points y₁, . . . , yₙ at detection location 1 of the blood vessel. Detected signals from multiple detection points are used as the multi-output signals at this location, i.e., multiple point pulse waves, denoted by Y₁(y₁, . . . , yₙ,e^{jω}). Set a plurality of detection points z₁, . . . , zₜ at detection location 2 of the blood vessel. Detected signals from multiple detection points are used as the multi-output signals at this location, i.e., multiple point pulse waves, denoted by Z₂(z₁, . . . , zₜ,e^{jω}). The vascular conduction characteristics between detection location 0 and detection location 1, between detection location 0 and detection location 2, and between detection location 1 and detection location 2, are denoted by H₀₁(e^{jω}), H₀₂(e^{jω}), and H₁₂(e^{jω}), respectively. Then

$${H}_{12} ( {e}^{j ω} ) = \frac{{Z}_{2} ( {z}_{1} , … , {z}_{t} , {e}^{j ω} )}{{Y}_{1} ( {y}_{1} , … , {y}_{n} , {e}^{j ω} )} = \frac{{Z}_{2} ( {z}_{1} , … , {z}_{t} , {e}^{j ω} ) / X ( {x}_{1} , … , {x}_{m} , {e}^{j ω} )}{{Y}_{1} ( {y}_{1} , … , {y}_{n} , {e}^{j ω} ) / X ( {x}_{1} , … , {x}_{m} , {e}^{j ω} )} = \frac{{H}_{0 \; 2} ( {e}^{j ω} )}{{H}_{01} ( {e}^{j ω} )} \qquad ( 1 )$$

Therefore, through the detection of myocardial point fluctuations and point pulse waves of the blood vessel, based on the cascade feature of the blood vessel the conduction characteristics of a certain section of the blood vessel can be obtained, which is helpful for localization of vascular disease.

FIG. 6 is a workflow diagram of the invention. The concrete steps are as follows.

Step 1: Parameter setting of the device.

The set parameters include: locations of the detection points, parameters of the broadband detection signal, parameters of the inversion unit, parameters of the conduction characteristic identification unit, parameters of the feature extraction unit, parameters of the communication unit, and parameters for displaying detection results;

Step 2: Automatic detection of device status.

Device status detected in this step includes: connection status of the probe array of flexible detection terminals, connection status of the flexible detection terminal and the control module, connection status of the control module and the computing module, connection and online status of the communication unit, power capacity status of flexible detection terminals;

Step 3: Generation of the broadband detection signal.

The broadband signal generation unit of the control module generates the broadband detection signal according to the parameters of the broadband detection signal set in Step 1;

Step 4: Ultrasonic wave transmission.

The communication unit of the flexible detection terminal is responsible for receiving the broadband detection signal generated by the control module. The received signal goes through the amplifier and D/A converter in the transmission processing unit, and is converted by the transmission probe into ultrasonic waves, which are then emitted;

Step 5: Ultrasonic echo reception.

The emitted ultrasonic waves are reflected and form ultrasonic echo when encountering the heart muscle or vascular walls. Part of the ultrasonic echo is received by the adjacent ultrasonic probes and the received echo signal is stored in the data buffering unit after going through the amplifier and A/D converter in the reception processing unit and then sent to the control module through the communication unit;

Step 6: Inversion of point fluctuation/point pulse wave;

Perform time and spatial time-domain/frequency-domain inversion of the positions and their variation of multiple detection points of the heart muscle/vascular walls in the detection area using frequency estimation algorithms and geometric principles, and obtain multidimensional time-domain/frequency-domain characteristics of point fluctuations of the heart muscle or point pulse waves of vascular walls;

Step 7: Identification and decomposition of the conduction characteristics.

Take the multiple point fluctuations of the heart muscle as the input and the multiple point pulse waves of the vascular wall as the output and identify the vascular conduction characteristics between the multi-input signal and the multi-output signal using digital signal processing methods. Then obtain the conduction characteristics of a certain section of the blood vessel by decomposing the vascular conduction characteristics based on the cascade feature of the blood vessel;

Step 8: Feature extraction.

By analyzing the vascular conduction characteristics in the time domain/frequency domain, obtain the time-domain/frequency-domain conduction feature of a certain section of the blood vessel;

Step 9: Result storing and classification.

According to the vascular conduction feature, judge the condition of the blood vessel detected and display it on the human-computer interaction platform. Also store the classification feature database, detection location, vascular conduction features, myocardial point fluctuations, point pulse waves, the patient's symptoms and age, etc., in the storage unit and the detection results are divided into the following three categories and processed separately.

(1) with disease symptoms. The detection results in this category indicate that a certain section of the blood vessel possesses relevant pathological features and matches the feature information in the classification feature database. The feature information of the vascular conduction characteristics will be retained in the feature database of the corresponding symptom in the storage unit;

(2) no disease symptoms. The detection results in this category indicate that a certain section of the blood vessel does not possess relevant pathological features and the data in the storage unit are directly discarded;

(3) suspicious disease symptoms. The detection results in this category cannot provide a definite diagnosis of the vascular condition. Such feature data are stored in the storage unit and when the detection device is available or the test is completed, the testing staff can choose whether to perform repeated tests and more accurate data processing.

It is to be understood that various other adaptations and modifications can be made within the spirit and scope of the invention. Therefore, it is the object of the appended claims to cover all such variations and modifications as come within the true spirit and scope of the invention.

We claim:

1. A method for detecting the vascular condition based on point fluctuation conduction characteristics of the heart, wherein the detection area of the heart muscle or vascular walls is discretized into a plurality of detection points, and through continuous active detection of the positions and their variations of the detection points, multiple point fluctuations of the heart muscle and multiple point pulse waves of the vascular wall can be obtained.

2. A method for detecting the vascular condition based on point fluctuation conduction characteristics of the heart, wherein flexible detection terminals are placed at multiple detection locations of the heart, artery, and vein;
wherein each flexible detection terminal performs multi-point detection for each detection location through ultrasonic transmission and reception;
wherein by taking the multiple point fluctuations of the heart muscle as the multiple input signals of the vascular system and the multiple point pulse waves of a certain detection location of the blood vessel as the multiple output signals of the vascular system, the vascular conduction characteristics can be identified using digital signal processing methods;
wherein the conduction characteristics of a certain section of the blood vessel can be obtained by decomposing the vascular conduction characteristics at different locations based on the cascade feature of the blood vessel; and
wherein through feature extraction and feature comparison, the vascular condition and the corresponding symptoms can be diagnosed and disease prediction and curative effect tracking can also be realized.

3. A device that detects vascular condition based on point fluctuation conduction characteristics of the heart, comprising
a plurality of flexible detection terminals with ultrasonic transceiving capability;
a control module; and
a computing module,
wherein the flexible detection terminal is connected with the control module;
wherein the control module is connected with the computing module;
wherein the flexible detection terminal is responsible for driving and transmitting the ultrasonic detection signal, receiving and preprocessing the ultrasonic echo signal, and data communications;
wherein the control module is responsible for setting and controlling detection parameters of the whole device, generating detection signals, storing detection data, comparing vascular conduction characteristics, data communications, and human-computer interaction; and
wherein the computing module is responsible for inversion of point fluctuation and pulse waves, identification of vascular conduction characteristics, extraction of vascular features, and data communications.

4. The flexible detection terminal of claim 3, comprising
an ultrasonic transceiver probe array;
a transmission processing unit;
a reception processing unit; and
a communication unit,
wherein all of them are fixed on a flexible circuit board and packaged using sound transparent and waterproof material;
wherein the flexible detection terminal is fixed by tape on the external skin close to the heart or the vascular wall and firmly against the skin using couplant;
wherein the ultrasonic transceiver probe array is composed of multiple ultrasonic probes that are arranged according to a certain rule and form an array, wherein one of the probes is responsible for ultrasonic emission and the other probes are responsible for ultrasonic echo reception;
wherein the transmission processing unit performs amplification and D/A conversion on the signal received from the control module according to the set parameters and through the transmission probe the obtained electrical signal is converted into an ultrasonic signal, which is then emitted;
wherein the reception processing unit performs amplification, A/D conversion and data buffering on the received signal according to the set parameters; and
wherein the communication unit is responsible for the data and control information communication between the flexible detection terminal and the control module using the wired/wireless communication method.

5. The control module of claim 3, comprising
a broadband signal generation unit;
a master controller;
a storage unit;
a feature comparison unit;
a human-computer interaction unit;
a communication unit; and
a data buffering unit,
wherein the broadband signal generation unit is responsible for generating and driving the broadband detection signal, which is sent to different flexible detection terminals through the communication unit;
wherein the master controller is responsible for scheduling among different units to ensure the normal operation of the entire system;
wherein the storage unit is in charge of storing the classification feature database, detection locations, vascular conduction features, myocardial point fluctuations, point pulse waves, the patient's symptoms and age, etc.;
wherein the feature comparison unit is responsible for carrying out comparison between the vascular conduction characteristics and the feature database;
wherein the human-computer interaction unit is responsible for human-computer interaction and can be used for the parameter setting, the input of the control command and the output of the error information;
wherein the communication unit is responsible for the data and control information communication between the flexible detection terminal and the control module as well as between the control module and the computing module using the wired/wireless communication method; and
wherein the data buffering unit is responsible for caching ultrasound echo signals and control parameters.

6. The computing module of claim 3, comprising
an inversion unit;
a conduction characteristic identification unit;
a feature extraction unit; and
a communication unit,
wherein based on the ultrasonic echo signals received by the reception probes of the flexible detection terminal, the inversion unit performs time and spatial time-domain/frequency-domain inversion of the positions and their variations of multiple detection points of the heart muscle or vascular wall in the detection area using frequency estimation algorithms and geometric principles, and obtains multidimensional time-domain/frequency-domain characteristics of multiple point fluctuations of the heart muscle or multiple point pulse waves of the vascular wall;
wherein the conduction characteristic identification unit takes the multiple point fluctuations of the heart muscle as the input and the multiple point pulse waves of the vascular wall as the output, identifies the vascular conduction characteristics between the multi-input signal and the multi-output signal using digital signal processing methods, and then decomposes the vascular conduction characteristics based on the cascade feature of the blood vessel to obtain the conduction characteristics of a certain section of the blood vessel;
wherein the feature extraction unit is responsible for extracting the feature of the vascular conduction characteristics and then obtaining the conduction feature of a certain section of the blood vessel; and
wherein the communication unit is responsible for the data and control information communication between the computing module and the control module using the wired/wireless communication method.

7. The method for detecting the vascular condition based on point fluctuation conduction characteristics of the heart of claim 2, includes the following steps:
step 1: parameter setting of the device, wherein
the set parameters include: locations of the detection points, parameters of the broadband detection signal, parameters of the inversion unit, parameters of the conduction characteristic identification unit, parameters of the feature extraction unit, parameters of the communication unit, and parameters for displaying detection results;
step 2: automatic detection of device status, wherein
device status detected in this step includes: connection status of the probe array of flexible detection terminals, connection status of the flexible detection terminal and the control module, connection status of the control module and the computing module, connection and online status of the communication unit, power capacity status of flexible detection terminals;
step 3: generation of the broadband detection signal, wherein
the broadband signal generation unit of the control module generates the broadband detection signal according to the parameters of the broadband detection signal set in Step 1;
step 4: ultrasonic wave transmission, wherein
the communication unit of the flexible detection terminal is responsible for receiving the broadband detection signal generated by the control module; and
wherein the received signal goes through the amplifier and D/A converter in the transmission processing unit, and is converted by the transmission probe into ultrasonic waves, which are then emitted;
step 5: ultrasonic echo reception, wherein
the emitted ultrasonic waves are reflected and form ultrasonic echo when encountering the heart muscle or vascular walls; and
wherein part of the ultrasonic echo is received by the adjacent ultrasonic probes and the received echo signal is stored in the data buffering unit after going through the amplifier and A/D converter in the reception processing unit and then sent to the control module through the communication unit;
step 6: inversion of point fluctuation/point pulse wave, wherein
perform time and spatial time-domain/frequency-domain inversion of the positions and their variation of multiple detection points of the heart muscle/vascular walls in the detection area using frequency estimation algorithms and geometric principles, and obtain multidimensional time-domain/frequency-domain characteristics of point fluctuations of the heart muscle or point pulse waves of vascular walls;
step 7: identification and decomposition of the conduction characteristics, wherein
take the multiple point fluctuations of the heart muscle as the input and the multiple point pulse waves of the vascular wall as the output and identify the vascular conduction characteristics between the multi-input signal and the multi-output signal using digital signal processing methods; and
wherein obtain the conduction characteristics of a certain section of the blood vessel by decomposing the vascular conduction characteristics based on the cascade feature of the blood vessel;
Step 8: feature extraction, wherein
the time-domain/frequency-domain conduction feature of a certain section of the blood vessel can be obtained by analyzing the vascular conduction characteristics in the time domain/frequency domain;
step 9: result storing and classification, wherein
according to the vascular conduction feature, judge the condition of the blood vessel detected and display it on the human-computer interaction platform;
store the classification feature database, detection locations, vascular conduction features, myocardial point fluctuations, point pulse waves, the patient's symptoms and age, etc., in the storage unit; and
wherein the detection results are divided into the following three categories and processed separately:
(1) with disease symptoms, wherein
the detection results in this category indicate that a certain section of the blood vessel possesses relevant pathological features and matches the feature information in the classification feature database; and
wherein the feature information of the vascular conduction characteristics will be retained in the feature database of the corresponding symptom in the storage unit;
(2) no disease symptoms, wherein
the detection results in this category indicate that a certain section of the blood vessel does not possess relevant pathological features and the data in the storage unit are directly discarded;
(3) suspicious disease symptoms, wherein
the detection results in this category cannot provide a definite diagnosis of the vascular condition; and
wherein such feature data are stored in the storage unit and when the detection device is available or the test is completed, the testing staff can choose whether to perform repeated tests and more accurate data processing.
